(19) United States
(12) Patent Application Publication
(10) Pub. No.: US 2012/0078059 A1
(43) Pub. Date: Mar. 29, 2012

(54) MINIMALLY INVASIVE SUCTION RETRACTOR

(75) Inventors: Miguelangelo J. Perez-Cruet, Bloomfield, MI (US); John R. Pepper, Cheshire, CT (US)

(73) Assignee: MI4SPINE, LLC, Bloomfield Village, MI (US)

(21) Appl. No.: 12/981,019

(22) Filed: Dec. 29, 2010

Related U.S. Application Data
(60) Provisional application No. 61/385,771, filed on Sep. 23, 2010.

(51) Int. Cl. A61B 1/32

(52) U.S. Cl. 600/210

(57) ABSTRACT

A suction retractor including an elongated tube that is operable to be inserted through a tubular retractor used in minimally invasive surgical procedures. One end of the tube is coupled to a handle having a chamber and the other end of the tube is coupled to a curved retractor portion, where the retractor portion allows the surgeon to hold back various anatomies during the surgical procedure. Thus, the suction retractor provides two functions that may otherwise require two different instruments. The retractor portion includes a series of parallel groves that allow liquids, such as blood, to flow along the retractor portion and into a suction port at the end of the tube. A suction device can be coupled to an outlet port in the handle that causes the blood and other surgical material to be sucked through the tube and out of the handle.

### CROSS REFERENCE TO RELATED APPLICATIONS

This application claims the benefit of the priority date of U.S. Provisional Patent Application No. 61/385,771, titled Minimally Invasive Suction Retractor, filed Sep. 23, 2010.

### BACKGROUND OF THE INVENTION

1. Field of the Invention

This invention relates generally to a surgical retractor that provides suction and, more particularly, to a surgical retractor that provides suction and has specific application for minimally invasive spinal surgical procedures.

2. Discussion of the Related Art

In an attempt to preserve normal anatomical structures during spine surgery, minimally invasive surgical procedures have been devised. These procedures include lumbar laminectomy for stenosis, cervical laminectomy, lumbar disectomy, spinal fusion, etc. Such procedures typically involve the use of a series of muscle dilators that separate the muscle fibers of the spine to create a pathway to the spine. A Kirschner (K-wire) is initially introduced through a small incision and directed towards the spinal pathology. The position of the K-wire is visualized by a fluoroscopic imaging system to identify its location. An initial narrow diameter muscle dilator is passed over the K-wire, and the K-wire is removed and subsequent larger muscle dilators are continually passed. When the opening is large enough, an access tube or retractor is positioned around the last muscle dilator through which the surgery is performed. The inner sequential muscle dilators are then removed allowing the surgeon to operate through the tubular retractor. The retractors come in a variety of lengths and diameters for different patients and procedures.

The spinal cord and spinal nerves are covered by a watertight sac of tissue, referred to as the dura. The dura is sometimes injured during spinal surgical procedures, typically referred to a dural tear. A dural tear should be surgically repaired during the surgical procedure to prevent spinal fluid from leaking out of the sac. Because the tubular retractor through which the surgeon is performing the surgical procedure is quite narrow, the ability to perform a delicate suturing process to repair the tear is difficult.

There are many surgical instruments that are specially designed for minimally invasive spinal surgical procedures that allow the surgeon to operate through the tubular retractor. These various instruments include retractors, suction devices, drills, etc. that allow the surgeon to perform the surgical procedure in the minimal space provided. Often, more than one of these instruments needs to be inserted through the tubular retractor at the same time to perform a particular procedure. Further, the various instruments that are used during the surgical procedure may be harmful to certain anatomies, such as the dural sac, nerves, etc. Therefore, it is often necessary to use a retractor to hold back such anatomies while the surgeon is performing a certain operation.

### SUMMARY OF THE INVENTION

In accordance with the teachings of the present invention, a suction retractor is disclosed that has particular application for minimally invasive spinal surgical procedures. The retractor includes an elongated tube that is operable to be inserted through a tubular retractor used in minimally invasive surgical procedures. One end of the tube is coupled to a handle having a chamber and the other end of the tube is coupled to a curved retractor portion, where the retractor portion allows the surgeon to hold back various anatomies during the surgical procedure. Thus, the suction retractor provides two functions that may otherwise require two different instruments. The retractor portion includes a series of parallel groves that allow liquids, such as blood, to flow along the retractor portion and into a suction port at the end of the tube. A suction device can be coupled to an outlet port in the handle that causes the blood and other surgical material to be sucked through the tube and out of the handle.

Additional features of the present invention will become apparent from the following description and appended claims, taken in conjunction with the accompanying drawings.

### BRIEF DESCRIPTION OF THE DRAWINGS

FIG. 1 is a perspective view of a suction retractor;

FIG. 2 is a cross-sectional view of the suction retractor shown in FIG. 1;

FIG. 3 is a perspective view of a retraction end of the suction retractor shown in FIG. 1; and

FIG. 4 is a perspective view of a suction retractor having an oppositely oriented retractor portion than the retractor shown in FIG. 1.

### DETAILED DESCRIPTION OF THE EMBODIMENTS

The following discussion of the embodiments of the invention directed to a suction retractor for minimally invasive surgical procedures is merely exemplary in nature, and is no way intended to limit the invention or its application or uses. For example, the suctionary retractor discussed below has particular application for minimally invasive surgical procedures performed through a tubular retractor. However, as will be appreciated by those skilled in the art, the retractor disclosed herein may have application for other surgical procedures.

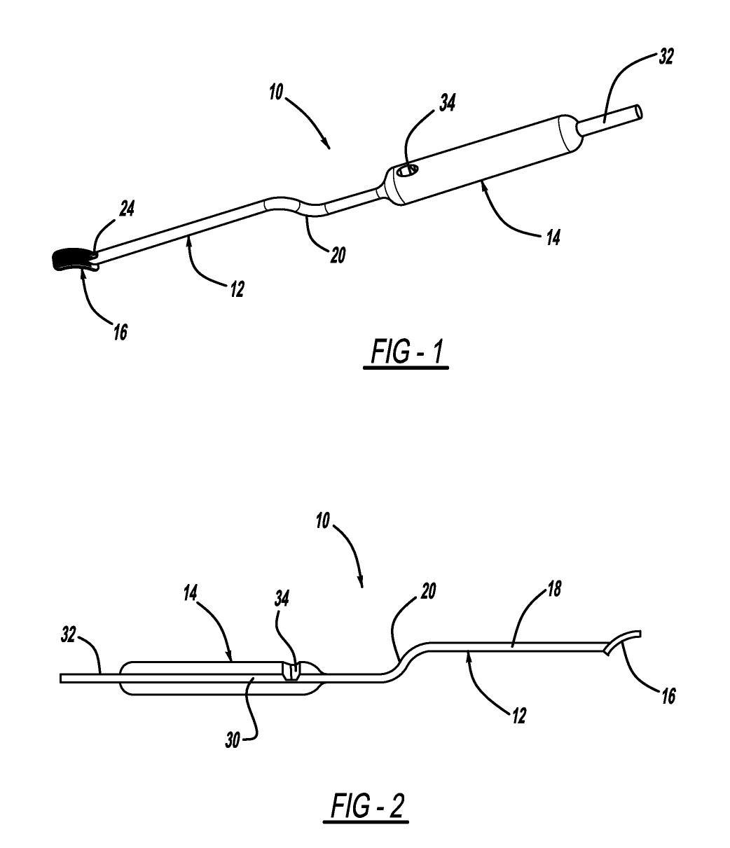

FIG. 1 is a perspective view and FIG. 2 is a cross-sectional view of a suction retractor 10 that has particular application for minimally invasive spinal surgical procedures being performed through a tubular retractor of the type discussed above. The suction retractor 10 includes an elongated tube 12 having an internal bore 18. A cylindrical handle 14 is coupled to one end of the tube 12 and a retractor portion 16 is coupled to an opposite end of the tube 12. The tube 12 has an offset curved section 20 that provides the surgeon with greater visibility of the retractor portion 16 when he is holding the handle 14 during the surgical procedure. In one non-limiting embodiment, the tube 12 has a 10 or 12 French sized diameter suitable for the minimally invasive surgical procedure. As will be discussed below, the retractor portion 16 allows the surgeon to retract various anatomies and structures during the surgical procedure, for example, the dura sac, and the suction capability of the suction retractor 10 allows blood and other materials to be sucked from the surgical area through the tube 12 during the surgical procedure.

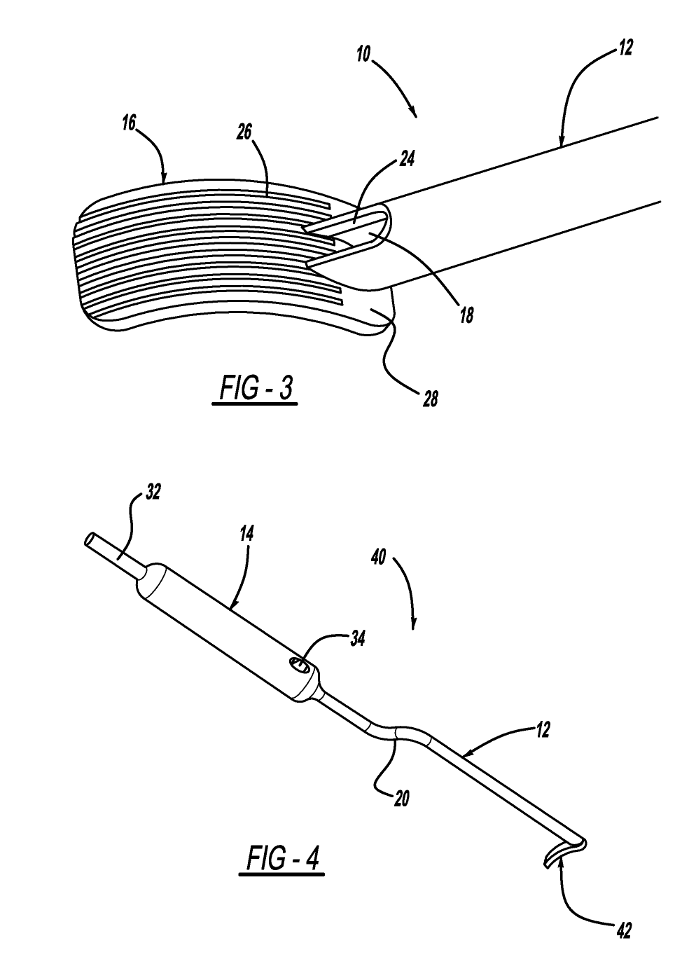

FIG. 3 is a perspective view of an end of the retractor 10 including the retractor portion 16. The tube 12 includes a suction inlet port 24 proximate the retractor portion 16 and extending part way up the tube 12 from the retractor portion 16. The end of the tube 12 is mounted to one end of the retractor portion 16 by any suitable technique, such as gluing or welding, so that the inlet port 24 is open to the surgical area, as shown. The retractor portion 16 has a curved configuration, which may have any suitable degree of curvature for a particular application, such as a contour that would match the dura of the spine. The retractor portion 16 includes a series of parallel grooves 26 formed in a top surface 28 of the retractor portion 16 that help blood and other material to be guided to the port 20 along the top surface 28 of the retractor portion 16. Thus, the retractor portion 16 can be placed on the dura, or other anatomy, to hold it away from the surgical operating area and the inlet port 24 can be used to draw blood and other materials from the surgical area.

The handle 14 includes an internal chamber 30 that is in fluid communication with the bore 18 extending through the tube 12. The handle 14 also includes a suction outlet port 32 in fluid communication with the chamber 30 at an end of the handle 14 opposite to the tube 12 that is attachable to a suction hose (not shown) and a suitable suction pumping system of the type well known to those skilled in the art. When the suction hose is coupled to the suction outlet port 32, suction is provided to the surgical area to draw material through the inlet port 24. In this embodiment, the handle 14 includes a thumb vent 34 in fluid communication with the chamber 30 that allows the surgeon to control the suction, where suction is provided when the surgeon places his thumb over the vent 34 and suction is removed when the surgeon removes his thumb from the vent 34. The handle 14 is shown to have a cylindrical shape in this embodiment. However, as will be appreciated by those skilled in the art, the handle 14 can have any ergonomical shape suitable for the procedures being discussed herein. The tube 12 and the retractor portion 16 can be made of any material suitable for the purposes described herein, such as stainless steel, Peek, etc.

In the embodiment discussed above, the retractor portion 16 has a width of about 5 mm. In other designs, the width of the retractor portion 16 can be provided in different sizes for different applications, such as 10 mm and 2.5 mm, to provide a set of retractors.

The suction retractor 10 shows the retractor portion 16 oriented so that it extends away from the tube 12. This orientation of the retractor portion 16 may be suitable for many of the surgical procedures. However, for other surgical procedures, such as a contra lateral laminectomy, it may be desirable to position the retractor portion 16 in an opposite direction. To illustrate this embodiment, FIG. 4 shows a perspective view of a suction retractor 40 similar to the suction retractor 10, where like elements are identified by the same reference numeral. In this embodiment, the retractor portion 16 has been replaced with a retractor portion 42 that extends in an opposite direction relative to the tube 12, where the tube 12 is coupled to an end of the retractor portion 42 that is opposite to the retractor portion 16.

The foregoing discussion discloses and describes merely exemplary embodiments of the present invention. One skilled in the art will readily recognize from such discussion and from the accompanying drawings and claims that various changes, modifications and variations can be made therein without departing from the spirit and scope of the invention as defined in the following claims.

What is claimed is:

1. A suction retractor comprising:
an elongated tubular portion having an inlet suction port at one end and a bore extending therethrough;
a handle portion having an internal chamber, said handle portion being coupled to an end of the tubular portion opposite to the suction port so that the chamber and the bore are in fluid communication with each other, said handle portion further including an outlet suction port in fluid communication with the chamber; and
a curved retractor portion mounted to the end of the tubular portion including the inlet suction port and opposite from the handle portion so that the inlet suction port is in fluid contact with a top surface of the retractor portion, wherein material that enters the inlet suction port is able to flow through the bore, into the chamber of the handle and out of the outlet suction port.

2. The retractor according to claim 1 wherein the top surface of the retractor portion includes a plurality of parallel grooves.

3. The retractor according to claim 1 wherein the tubular portion includes a curved section that causes the handle portion to be offset relative to the retractor portion.

4. The retractor according to claim 1 wherein the retractor portion is curved to match a dura of a human spine.

5. The retractor according to claim 1 wherein the width of the retractor portion is selected from the group comprising 2.5 mm, 5 mm and 10 mm.

6. The retractor according to claim 1 wherein the handle portion includes a vent in fluid communication with the chamber.

7. The retractor according to claim 1 wherein the outlet suction port is in an end of the handle portion opposite to the tubular portion.

8. The retractor according to claim 1 wherein the tubular portion is coupled to an end of the retractor portion so that an opposite end of the retractor portion extends away from the tubular portion.

9. The retractor according to claim 1 wherein the tubular portion is coupled to an end of the retraction portion so that an opposite end of the retractor portion extends back towards the tubular portion.

10. A suction retractor comprising:
a hollow extended portion having an inlet suction port at one end;
a handle portion having an internal chamber, said handle portion being coupled to an end of the extended portion opposite to the inlet suction port so that the chamber and the extended portion are in fluid communication with each other, said handle portion further including an outlet suction port in fluid communication with the chamber; and
a retractor portion mounted to the end of the extended portion including the suction port and opposite from the handle portion, wherein material that enters the inlet suction port is able to flow through the extended portion into the chamber in the handle and out of the outlet suction port.

11. The retractor according to claim 10 wherein the retractor portion is curved.

12. The retractor according to claim 11 wherein the retractor portion is curved to match a dura of a human spine.

13. The retractor according to claim 10 wherein a top surface of the retractor portion includes a plurality of parallel grooves.

14. The retractor according to claim 10 wherein the extended portion includes a curved section that causes the handle portion to be offset relative to the retractor portion.

15. The retractor according to claim 10 wherein the width of the retractor portion is selected from the group comprising 2.5 mm, 5 mm and 10 mm.

16. The retractor according to claim 10 wherein the handle portion includes a vent in fluid communication with the chamber.

17. The retractor according to claim 10 wherein the outlet suction port is in an end of the handle portion opposite to the extended portion.

18. The retractor according to claim 10 wherein the extended portion is coupled to an end of the retractor portion so that an opposite end of the retractor portion extends away from the extended portion.

19. The retractor according to claim 10 wherein the extended portion is coupled to an end of the retraction portion so that an opposite end of the retractor portion extends back towards the extended portion.

20. A surgical process comprising:
providing a suction retractor including an elongated tubular portion having an inlet suction port at one end and a bore extending therethrough, a handle portion having an internal chamber, said handle portion being coupled to an end of the tubular portion opposite to the suction port so that the chamber and the bore are in fluid communication with each other, said handle portion further including an outlet suction port in fluid communication with the chamber, and a curved retractor portion mounted to the end of the tubular portion including the inlet suction port and opposite from the handle portion so that the inlet suction port is in fluid contact with a top surface of the retractor portion, wherein material that enters the inlet suction port is able to flow through the bore, into the chamber of the handle and out of the outlet suction port; and
using the suction retractor during the surgical process to simultaneously remove the material from a surgical area and retract a structure from the surgical area.
